(19) United States
(12) Patent Application Publication
(10) Pub. No.: US 2008/0154300 A1
(43) Pub. Date: Jun. 26, 2008

(54) Surgical forceps

(76) Inventor: Noel Jabbour, Minneapolis, MN (US)

Correspondence Address: A and G Intellectual Property, PO Box 4254, Morgantown, WV, 26504, US

(21) Appl. No.: 12/001,731

(22) Filed: Dec. 12, 2007

Related U.S. Application Data
(60) Provisional application No. 60/875,264, filed on Dec. 15, 2006.

(51) Int. Cl. A61B 17/28

(52) U.S. Cl. 606/205

(57) ABSTRACT

The surgical forceps are comprised of three forceps arms pivotally attached to each other. The forceps may also contain distal grasping tips which are controlled by the proximal handles. The grasping tips create two or more grasping compartments which may be used to separately grasp lengths of suture. The controlling handles of each of the forceps arms may be individually controlled and may be secured together by a clasping mechanism. The surgical forceps may also be embodied by two central arms and one or more lateral arms which may contain distal grasping tips which are controlled by the proximal handles or controlling levers. The grasping tips of the arms may create two or more grasping compartments which may be used together or separately.

### CROSS-REFERENCE TO RELATED APPLICATIONS

This application claims priority of U.S. Provisional Application No. 60/875264.

### STATEMENT REGARDING FEDERALLY SPONSORED RESEARCH OR DEVELOPMENT

### REFERENCE TO SEQUENCE LISTING, A TABLE, OR A COMPUTER PROGRAM LISTING COMPACT DISC APPENDIX

Not Applicable

### BACKGROUND OF THE INVENTION

Surgical forceps are well known instruments used by surgeons for blunt dissection, clamping, and passing of objects such as lengths of suture in an operative field. Many variations of two-armed surgical forceps are used by operators for these purposes. Such forceps have a variety of names, including but not limited to, Kelly forceps, Kelly clamps, Rankin forceps, mosquito forceps, and mosquito clamps. In addition, some two-armed forceps are named for their angle, such as straight forceps or straight clamps, 30-degree forceps or 30-degree clamps, and 90-degree forceps or 90-degree clamps.

It is a common surgical technique to use such a forceps or clamp for blunt dissection in the operative field. The operator places a thumb into one finger-hole of one arm of the instrument and his fourth finger into the finger-hole of the opposite arm of the instrument. The distal tip of the instrument is placed in the desired area of the operative field, and the operator spreads the tissues apart by separating the two arms of the instrument; this motion spreads apart the opposing surfaces of the distal tip of the instrument.

Often, an operator approaches a tubular structure, such as an artery or vein, or section of tissue during the dissection that he may wish to ligate. In such instances, the following technique of external suture ligation is often utilized. Using surgical forceps, the operator dissects to the tubular structure and dissects under it. Once under the tubular structure, the operator spreads his thumb and fourth-finger apart to open the distal tip of the forceps. A length of suture is passed to the distal tip of the forceps, either by his other hand or by an assistant. The operator decreases the distance between his thumb and finger, thereby closing the distal tip of the forceps and grasping the length of suture. The operator pulls with distal tip of the forceps along with one end of the length of suture back out from under the tubular structure being careful to prevent the opposite end of the suture from passing under the tubular structure. The forceps is opened, and the suture, which is passing under the tubular structure, is either tied tightly against the tubular structure at this point in the process or both ends of the suture are held together and temporarily secured. The operator repeats the above process of dissecting under the vessel, opening the forceps, grasping the suture, closing the forceps, and passing the forceps and suture back under the tubular structure. The forceps is opened, and the suture is tied tightly against the tubular structure; if the suture that was initially passed was not previously tied, it is also tied at this point in the process. With the tubular structure tied tightly in two adjacent locations, the operator may now use a pair of scissors to cut in between the two knots and to trim the remaining length of sutures.

The above process of external suture ligation of tubular structures is used in a variety of operative procedures. The process has as an inherent disadvantage which is the requirement that an operator make two separate passes under the tubular structure in order to grasp two separate lengths of suture. Having to repeat the same operative process is time consuming, which translates to increased operator time, increased anesthesia time, and increased operating room usage. This clearly has both financial and patient-safety disadvantages. Also, since the operator must make separate dissection attempts under the tubular structure, it is not uncommon for the operator to be in a separate plane of dissection from one pass to the other. When both sides are tied and the center is cut, this can lead to bleeding or loss of other fluid contents, if a vessel or other structure is above the plane of dissection and has been tied off on one side and is below the plane of dissection and has not been tied off on the other side. This clearly has patient-safety implications as well as financial implications, as increased operating room time is needed to control the bleeding.

### BRIEF SUMMARY OF THE INVENTION

One embodiment can be as a surgical instrument that would allow for a single pass under the tubular structure in the process of dissection and external suture ligation reducing operative time by eliminating repetitive steps and eliminating the possibility of dissecting into separate planes on repeat passes.

A further embodiment can be as a method for the surgical instrument that would allow for a single pass under the tubular structure in the process of external suture ligation thereby reducing operative time by eliminating repetitive steps and eliminating the possibility of dissecting into separate planes on repeat passes.

Another embodiment can be as a surgical forceps containing at least three arms and at lest two grasping surfaces wherein the opposing surfaces of the distal tips may contain a textured surface to improve grasping. The three arms may be pivotally connected by a central rivet or screw. The three grasping tips are each attached by a lever arm to proximal control handles. The proximal control handles may be secured to adjacent control handles by clasp mechanisms.

These embodiments of the invention provide an inexpensive and simple solution for the current problems with external suture ligation of tubular structures. Further, the embodiments operate in a manner that is familiar to those who are accustomed to operating the currently available surgical forceps.

An alternative embodiment can be a lateral grasping tip may be connected via a crossing lateral arm to a contralateral controlling lever. The crossing lateral arm may be connected to the other arms via a central rivet or screw. A coil spring attaches to the crossing lateral arm and an adjacent arm such that the grasping surface made by the lateral grasping tip opens when the controlling lever is not secured to the adjacent controlling handle. The controlling lever may be secured to the adjacent controlling handle by clasp mechanisms.

Another alternative embodiment is that two lateral grasping tips may be connected via crossing lateral arms to contralateral controlling levers, such that there are four distal grasping tips. The two central grasping tips may each have textured lateral surfaces to improve grasping. The two lateral grasping tips may each have textured medial surfaces to improve grasping. The two central grasping tips may be attached by a lever arm to proximal control handles. The proximal control handles may be secured to each other by clasp mechanisms. The two lateral grasping tips may each be connected via a crossing lateral arm to a contralateral controlling lever. The crossing lateral arm may be connected to the other arms via a central rivet, screw, pin or any other connecting means known to one skilled within the art. Each of the lateral arms may contain a coiled spring attached to the crossing lateral arm and an adjacent arm such that the grasping surface made by the lateral grasping tip opens when the controlling lever is not secured to the adjacent controlling handle. Each controlling lever may be secured to the adjacent controlling handle by clasp mechanisms.

### BRIEF DESCRIPTION OF THE SEVERAL VIEWS OF THE DRAWING

FIG. 1 is a top view of the surgical forceps with the grasping tips in a closed position and the clasp mechanisms of the control handles in a locked position.

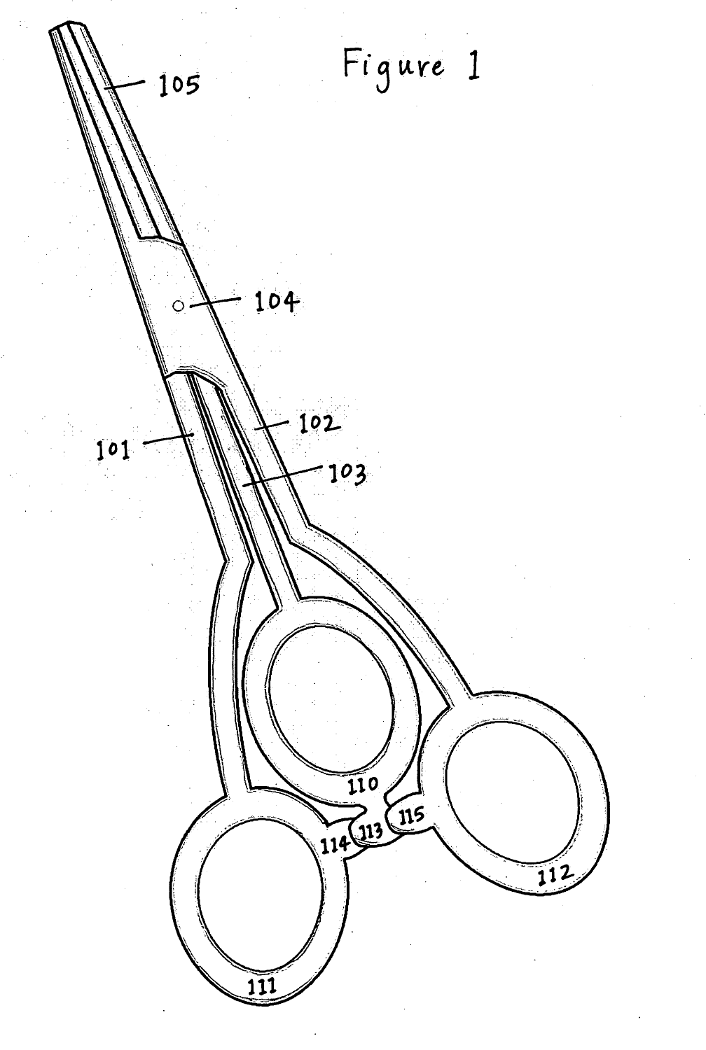

FIG. 2 is a top view of the surgical forceps, but with the right grasping tip in an opened position, the central and left grasping tips in a closed position, and the clasp mechanisms of the central control handle and the adjacent right lateral control handle in a locked position.

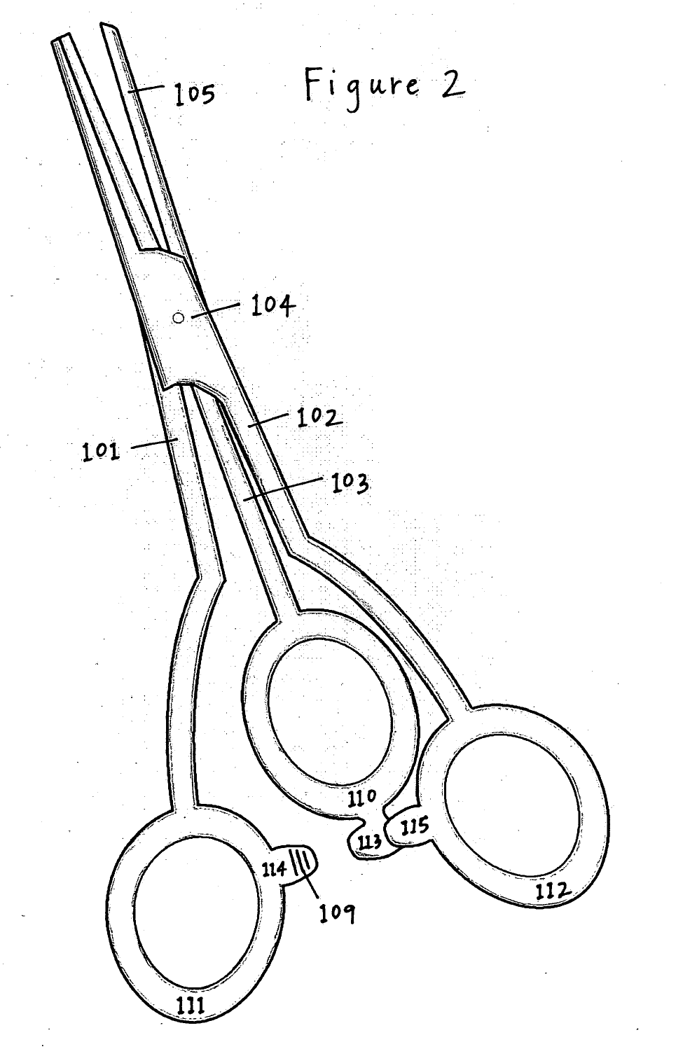

FIG. 3 is a top view of the surgical forceps, but with the left grasping tip in an opened position, the central and right grasping tip in a closed position, and the clasp mechanisms of the central control handle and the adjacent left lateral control handle in a locked position.

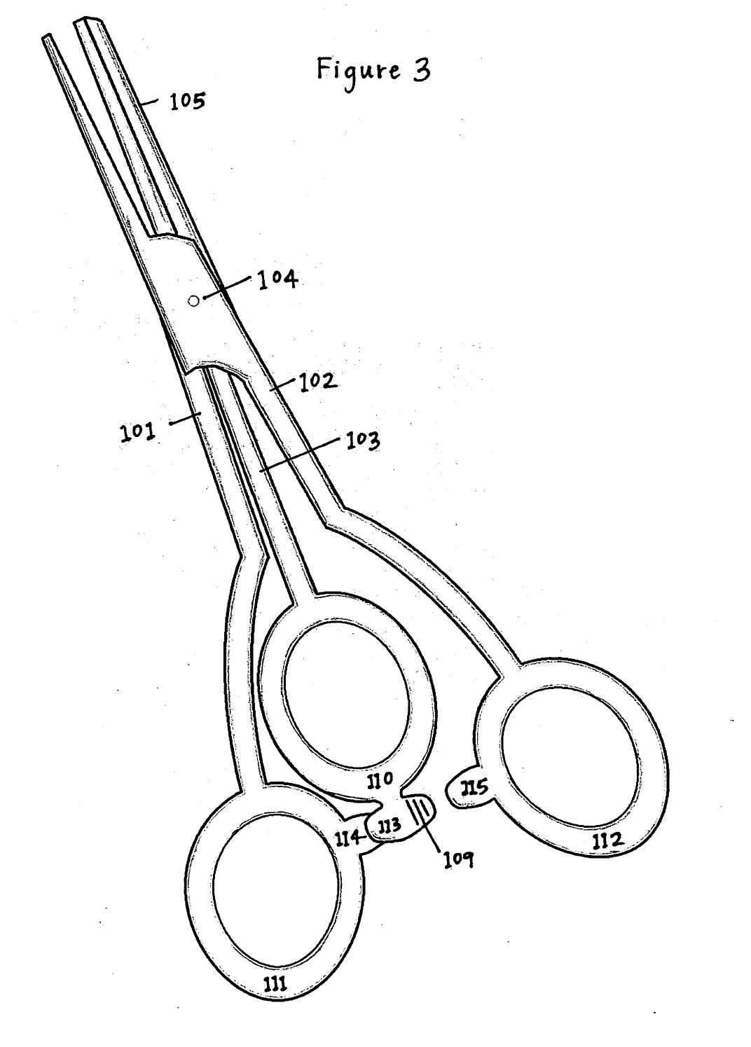

FIG. 4 is top view of the surgical forceps, but with all grasping tips in an open position and all clasp mechanisms in an unlocked position.

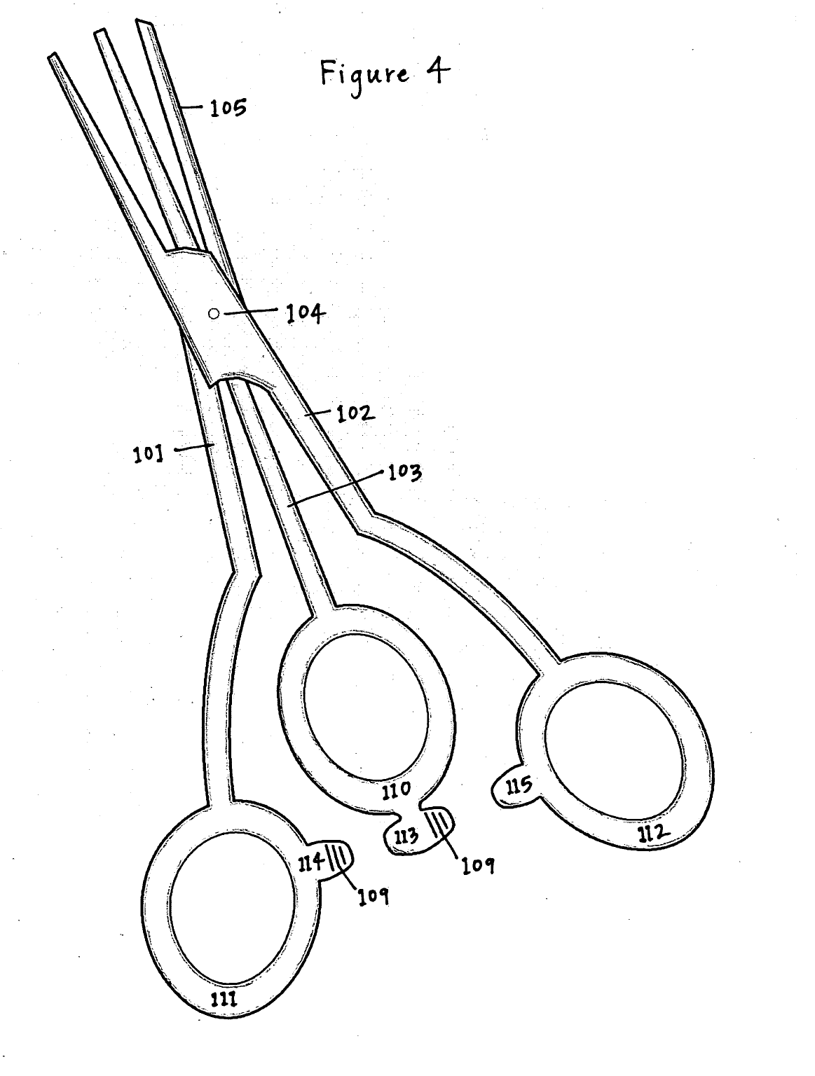

FIG. 5 is a front view of the proximal base of the surgical forceps showing one possible embodiment of the clasp mechanism, so that the clasping mechanism of the right lateral handle is superior to the clasping mechanism of the central handle, and the serrated projections of the right lateral handle clasp mechanism face downward while the serrated projections of the right side of the central handle claps mechanism face upwards.

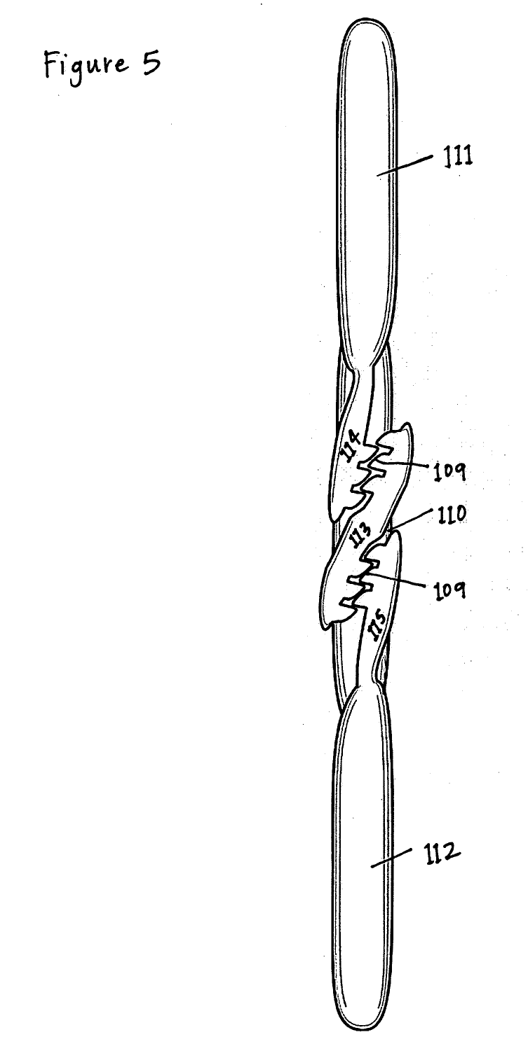

FIG. 6 is a front view of the proximal base of the surgical forceps showing one possible embodiment of the clasp mechanism, so that the clasping mechanism of the central handle is superior to the clasping mechanism of both lateral handles, with the serrated projections of the clasping mechanism of the lateral handles facing upward and the serrated projections of clasp mechanism of the central handle facing downward.

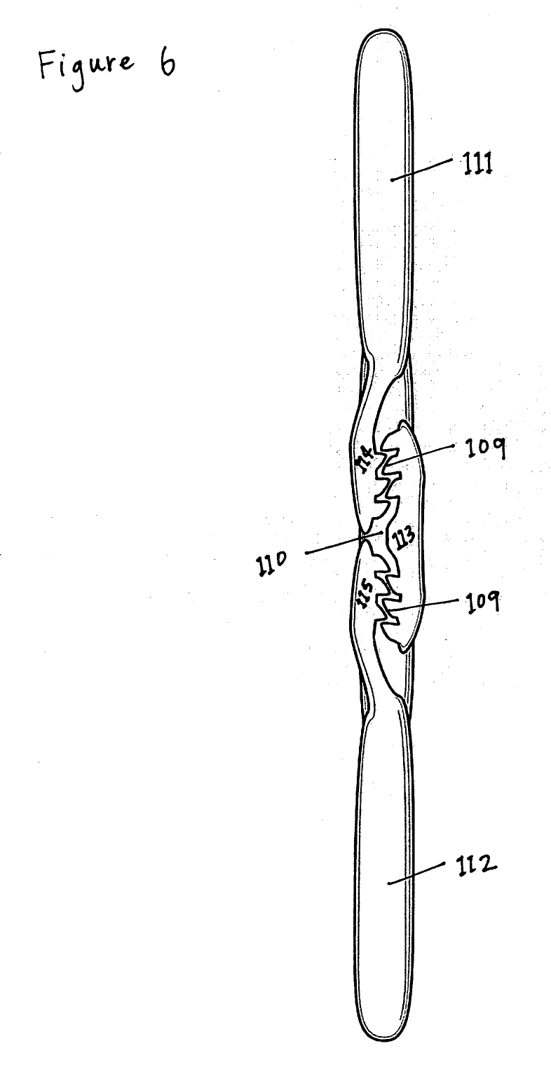

FIG. 7 is a top view of another embodiment of the invention, with a lateral grasping tip that is attached to a contralateral controlling lever, shown with the gasping tips in a closed position and the clamp mechanism of the contralateral controlling lever in a locked position relative to the adjacent control handle.

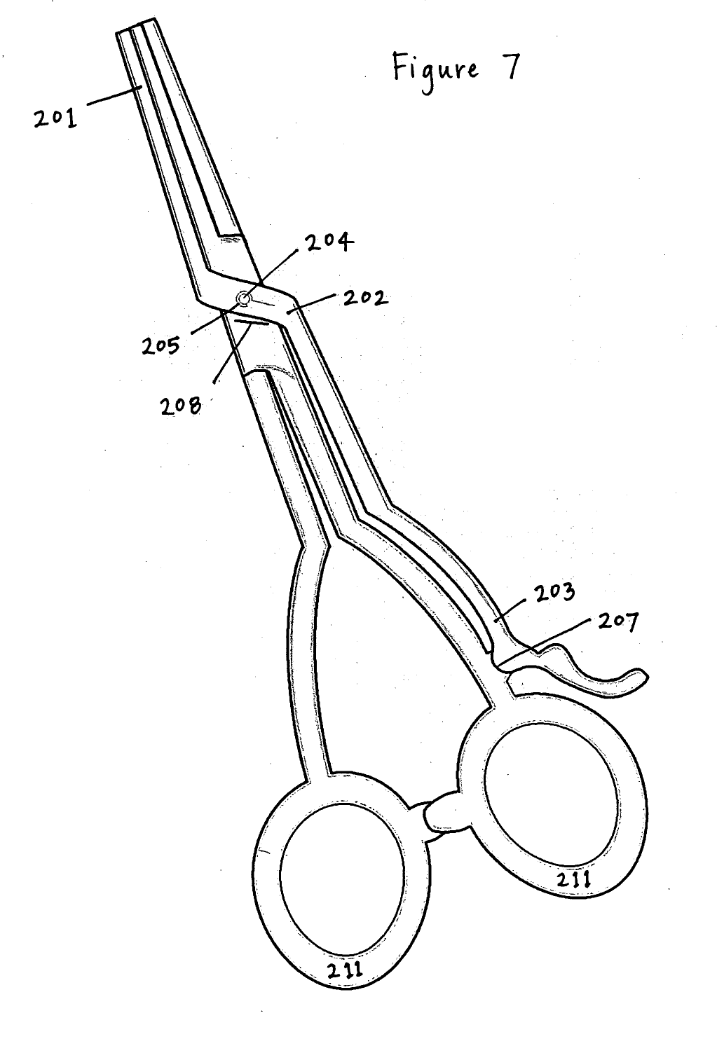

FIG. 8 is a top view of another embodiment of the invention, but with the lateral grasping tip in an open position and the contralateral controlling lever in an unlocked position relative to the adjacent control handle.

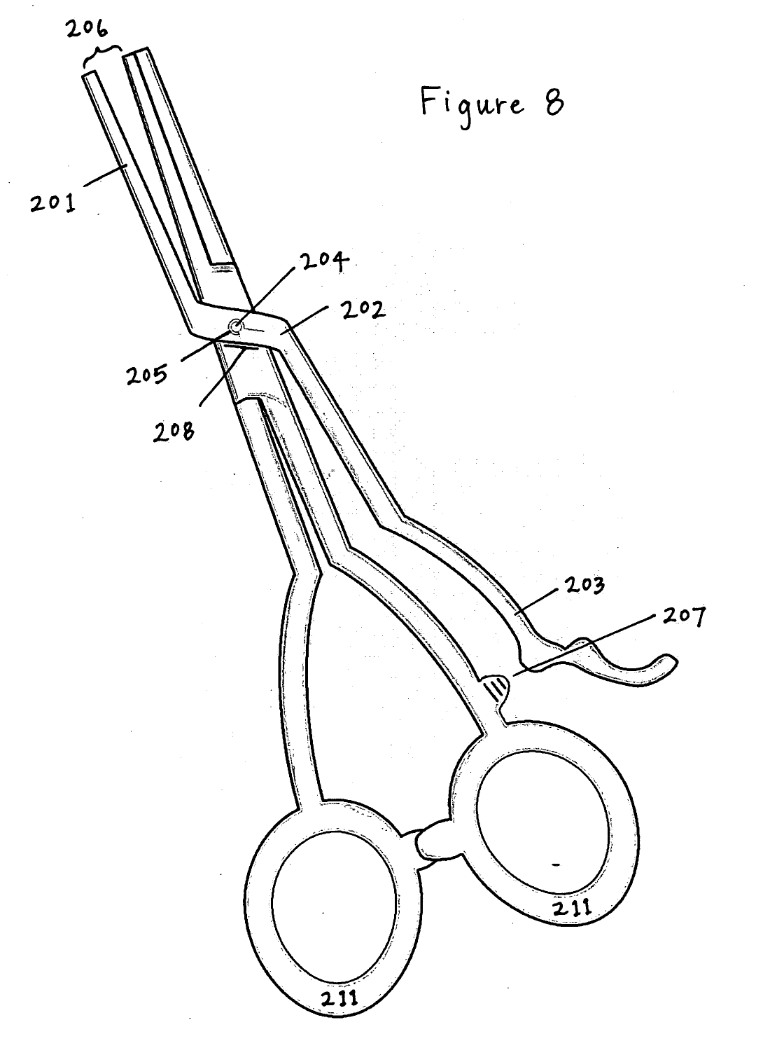

FIG. 9 is a top view of another embodiment of the invention, with two lateral grasping tips that are each attached to a contralateral controlling lever, shown with the gasping tips in a closed position and with each of the clamp mechanisms of the contralateral controlling levers in a locked position relative to their adjacent control handles.

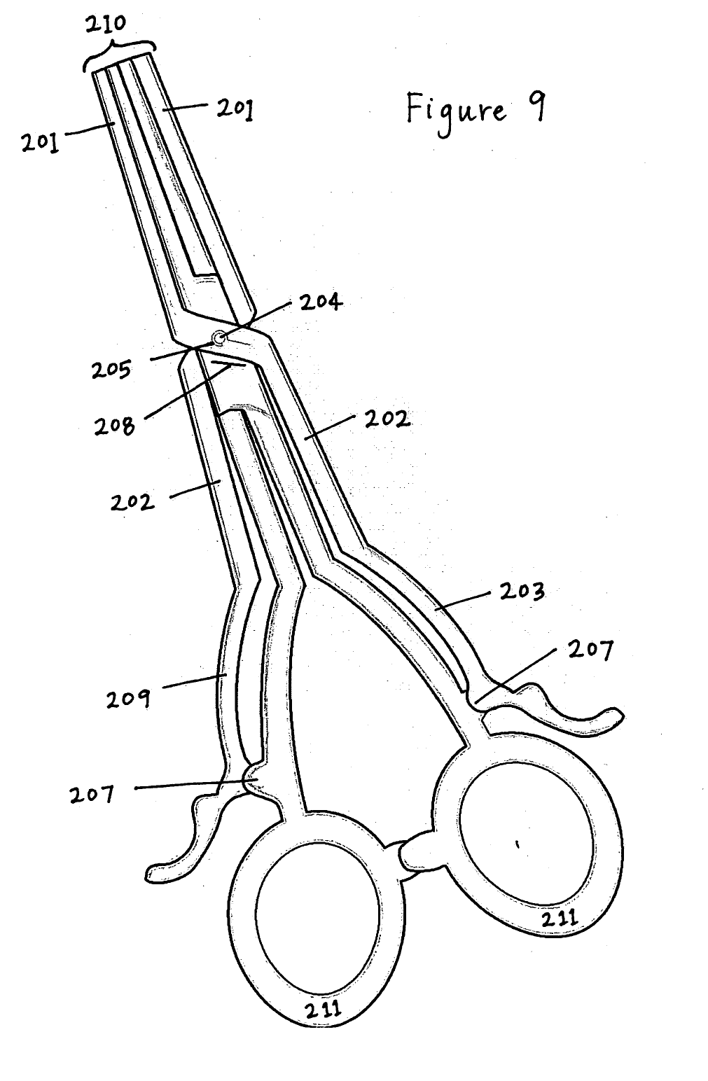

FIG. 10 is a view of some possible embodiments of the texture of the grasping surfaces and the shape of the distal tips of the surgical forceps.

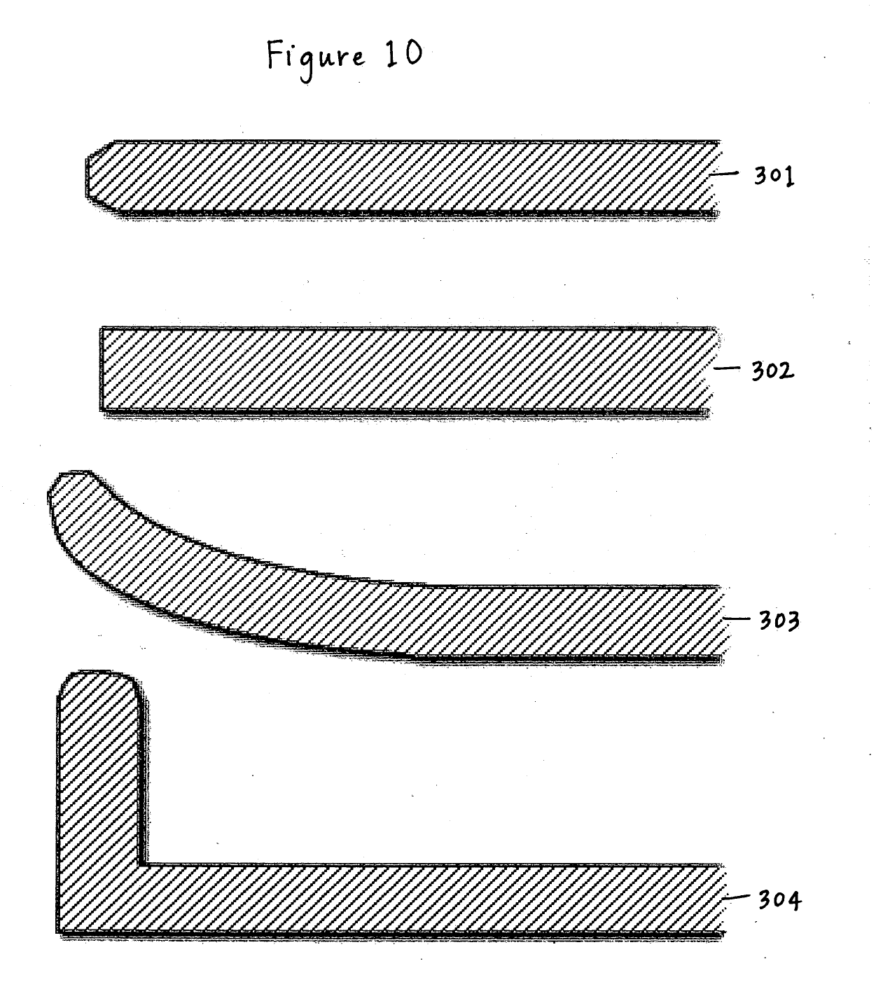

### DETAILED DESCRIPTION OF THE INVENTION

A first embodiment can be a surgical forceps which includes three arms: two lateral arms 101, 102 and one central arm 103. These arms are pivotally attached at a pivot point 104 by pivot attachment as is common to one skilled in the art such as by a screw, rivet, pin or any other attachment means known to one skilled within the art. The distal tip 105 of each lateral arm can contain a textured surface on the medial side of each arm to improve grasping. Both lateral surfaces of the distal tip of the central arm may contain a textured surface to improve grasping. The distal tip of the three arms may by manufactured in a variety of shapes 301-304 such as straight, rounded, or L-shaped. The selection of material for the manufacture of the instrument and the pattern for the textured surfaces may vary and may be left to those skilled in the surgical instrument art. The three arms together create two grasping compartments. Each grasping compartment is able to claim, grasp, a suture, or perform any function of a surgical forceps.

The grasping tip controls could be attached to any of the three handles. In one embodiment, the central grasping tip is attached to the central arm and can be controlled for grasping by a controlling mechanism such as if the grasping tip is attached by an arm to a handle wherein the handle could be circular for easy gripping. No matter what shape of handle is chosen the central handle could be positioned evenly with the other handles, positioned more proximally to the other handles or more distally positioned compared to the position of the other handles. The most proximal tip of the handles can include an extension which further can include a two-headed clasp mechanism 108. Each of the lateral grasping tips is each attached by a lever arm to a handle. The medial aspect of each of these handles can include a clasp mechanism extension to secure a clasping mechanism to the handle. These clasping mechanisms are well known to the art and include serrated projections 109 that engage the serrated projections of opposing clasp mechanisms in the closed positions.

In a central handle embodiment, the clasps and handles may be designed such that in the closed position, the clasping mechanism of the central handle 113 is superior to the clasping mechanism of the left lateral handle 114, and the serrated projections of the left-side of the central handle clasp mechanism face downward while the serrated projections of the left lateral handle claps mechanism face upwards. Similarly, in the closed position, the clasping mechanism of the right lateral handle 115 is superior to the clasping mechanism of the central handle 113, and the serrated projections 109 of the right lateral handle clasp mechanism face downward while the serrated projections 109 of the right side of the central handle claps mechanism face upwards.

It will be appreciated that this design for the clasping mechanisms and for the placement of the central handle will allow for an operator to individually unlock each clasping mechanism in order to freely operate each individual arm. The relationship of the clasping mechanism allows for the unlocking motions to be in a plane parallel to the natural motion of thumb extension and finger flexion for right-handed users. It will be appreciated also, that such a design allows for identical operation of the instrument regardless of sidedness of the instrument; that is, the orientation of the clasping mechanisms is preserved even when the instrument is flipped 180-degrees along its long axis.

In a central handle embodiment, the clasps and handles may alternatively be designed such that the clasping mechanism of the central handle 113 may be superior to the clasping mechanism of both lateral handles 114, 115. The serrated projections 109 of the clasping mechanism of the lateral handles would face upward, while the serrated projections of clasp mechanism of the central handle would face downward.

It will be appreciated that this design for the clasping mechanisms and for the placement of the central handle will allow for an operator to maintain the central handle attached to the right lateral handle during opening when the operator uses a finger flexion and thumb extension motion with a thumb in the left lateral handle and a finger in the right lateral handle.

The lateral arms of the instrument may be used in the usual manner as a conventional forceps for dissection, clamping, and passing of objects. An operator would place his thumb into one lateral handle and his fourth finger into the opposite lateral handle. The instrument of the invention can then be used in the usual manner with the central handle attached to either lateral arm, based on the operator's comfort. The additional grasping compartment allows freedom for the operator. If the central handle and lever arm are to be used, the operator may insert his second or third fingers into the handle, based on the operator's comfort. The clasping mechanisms may be unlocked by thumb extension and/or finger flexion, in the usual manner.

The instrument described above thus has three grasping arms which allow for two opposing grasping compartments at the distal tip of the instrument. Each of the arms may be operated independently by separate fingers in each handle. Because the instrument of the invention allows for two opposing grasping compartments, the instrument may be used in the following method for external suture ligation of tubular structures. An operator would grasp the instrument of the invention in the manner described above and would dissect using a spreading motion between one lateral handle and the opposite lateral handle; the central handle may be attached to either lateral handle by the clasp 113, 114, 115.

Using this technique, the operator would dissect under the tubular structure. After passing under the tubular structure with the distal tip of the instrument, the operator may open one of the grasping compartments. A length of suture may be inserted and the instrument returned to the closed and clasped position to grasp the end of the suture. While still in the same position under the tubular structure, the operator may open the other grasping compartment and a separate length of suture may be inserted. The instrument may be returned to the closed and clasped position. The instrument may be passed back under the tubular structure with each length of suture grasped in a separate grasping compartment, while being careful not to pull through the opposing ends of each of the lengths of suture. One grasping compartment may be opened, and the suture may be delivered to the operator's other hand or to an assistant. The suture may be used to make a tight knot against the tubular structure in the usual manner by the operator or an assistant. The other grasping compartment may then be opened, and the suture may be delivered to the operator's other hand or to an assistant. This suture may be used to make a tight knot against the tubular structure in the usual manner. The vessel may then be cut between these two adjacent ties, and the suture ends may be trimmed.

It will be appreciated that using the instrument of this invention with the above method successfully delivers two separate lengths of suture under the tubular structure, while keeping the lengths of suture from tangling together. This instrument and method, therefore eliminates the need for repeat dissection under the tubular structure. As described above, this may translate to reduced operative time and cost as well as improved patient-safety.

It will be appreciated that the illustrations and description pertain to a particular embodiment of this invention. This invention may be applied to all types of currently available surgical forceps and clamps which have lateral arms that are pivotally coupled at a pivot point. This invention may be applied to all angles of surgical forceps and clamps to provide the above advantages in a variety of cases for which different angles of surgical forceps or clamps are preferred.

Another embodiment of the present invention is that a lateral grasping tip 201 may be connected via a crossing lateral arm 202 to a contralateral controlling lever 203. The crossing lateral arm can be connected to the other arms via a central rivet, screw, pin or any other connecting means known to one skilled within the art 204. A spring mechanism such as a coil spring 205 attaches to the crossing lateral arm and an adjacent arm such that the grasping surface made by the lateral grasping tip opens 206 when the controlling lever is not attached to the adjacent controlling handle 211 and a grasping surface made by a lateral arm grasping tip closes when the controlling lever is pulled in the direction opposite of the adjacent handle. The controlling lever may be secured to the adjacent controlling handle 211 by clasp mechanisms 207. The central portion of the adjacent arm to which the lateral arm is attached, contains a small raised notch 208 which may be linear, just proximal to the proximal border of the crossing lateral arm, the function of which is to prevent the opening of the lateral arm beyond a predetermined and set angle.

The above described crossing-lateral-arm mechanism may be used on one side of the instrument only, or may be duplicated on the contralateral side of the instrument 209, such that there are four distal grasping tips 210, making three separate grasping compartments.

It will be appreciated that the spring mechanism of the above described crossing-lateral-arm mechanism is only one of many possible arrangements for spring mechanisms that may allow for the grasping surface made by the lateral grasping tip to open when the controlling lever is not secured to the adjacent controlling handle. One skilled within the art could readily change the spring to another spring type or any other similar mechanism known to one skilled within the art.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A surgical forceps comprising three forceps arms wherein the arms are two lateral arms and a central arm pivotally attached to one another by a pivot attachment wherein said forceps arms are comprised of a distal and a proximal end said distal end further comprising a grasping tip and proximal end further comprising a handle said surgical forceps further comprising two grasping compartments.

2. The surgical forceps of claim 1 wherein said pivotal attachment is one of a rivet, screw, or a pin.

3. The surgical forceps of claim 1 wherein said handles may be secured to one another by a clasp mechanism attached to said handles.

4. The surgical forceps of claim 1 wherein said clasp mechanisms are attached to said handles by a clasp mechanism extension.

5. The surgical forceps of claim 3 wherein two or more of said grasping tips are textured on at least one side of said distal end.

6. The surgical forceps of claim 1 further comprising a central handle embodiment wherein said forceps in a closed position have a central handle that is superior to the lateral handles.

7. The surgical forceps of claim 1 wherein said grasping tips may be one or more of pointed, squared off, or rounded at the most distal end.

8. The surgical forceps of claim 1 wherein said distal ends may be one or more of straight, rounded, or L-shaped.

9. A surgical forceps comprising two central arms pivotally attached to one another by a pivotal attachment and one or more lateral grasping arms wherein said one or more lateral arms are pivotally attached to the central arms by said pivotal attachment and all forceps arms further comprising a distal and a proximal end said distal end further comprising a grasping tip and proximal end further comprising a handle and said lateral arms further comprising a distal and a proximal end said distal end further comprising a grasping tip and proximal end further comprising a controlling lever said surgical forceps further comprising two or more grasping compartments.

10. The surgical forceps of claim 9 wherein said pivotal attachment is one of a rivet, screw, or pin.

11. The surgical forceps of claim 9 wherein said handles and controlling levers may be secured to one another by a clasp mechanism attached to said handles and controlling levers.

12. The surgical forceps of claim 11 wherein said clasp mechanisms are attached to said handles and controlling levers by a clasp mechanism extension.

13. The surgical forceps of claim 9 wherein two or more of said grasping tips are textured on at least one side of said distal end.

14. The surgical forceps of claim 9 wherein said lateral arms are attached to said central arm by a spring mechanism such that a grasping surface made by a lateral arm grasping tip opens when the controlling lever is not attached to the adjacent central handle such that a grasping surface made by a lateral arm grasping tip opens when the controlling lever is pulled in the direction of the adjacent central handle.

15. The surgical forceps of claim 14 wherein said spring mechanism is a coil spring.

16. The surgical forceps of claim 15 further comprising a small raised notch on the central portion of the central arm adjacent to the lateral arm to prevent the opening of the lateral arm beyond a predetermined set angle.

17. The surgical forceps of claim 9 wherein said grasping tips may be one or more of pointed, squared off, or rounded at the most distal end.

18. The surgical forceps of claim 9 wherein said distal ends may be one or more of straight, rounded, or L-shaped.

19. The surgical forceps of claim 9 further comprising a central handle embodiment wherein said forceps in a closed position have a central arm handle that is superior to the lateral controlling lever.

20. The surgical forceps of claim 11 further comprising a central handle embodiment wherein said forceps in a closed position have a central arm handle clasp mechanism that is superior to the controlling lever clasp mechanisms.
